(19) United States
(12) Patent Application Publication
(10) Pub. No.: US 2020/0298487 A1
(43) Pub. Date: Sep. 24, 2020

(54) STERILE ADDITIVE MANUFACTURING SYSTEM

(71) Applicant: Auregen BioTherapeutics SA, Geneva (CH)

(72) Inventors: James Alastair Devlin, Penn (GB); Matti Jaakko Johannes Kesti, Zurich (CH)

(73) Assignee: Auregen BioTherapeutics SA, Geneva (CH)

(21) Appl. No.: 16/765,662

(22) PCT Filed: Nov. 29, 2018
(86) PCT No.: PCT/IB2018/059476

§ 371 Date: May 20, 2020

Related U.S. Application Data
(60) Provisional application No. 62/592,202, filed on Nov. 29, 2017.
(60) Provisional application No. 62/652,757, filed on Apr. 4, 2018.

(51) Int. Cl. B29C 64/255; A61L 27/20; B29C 64/209; A61L 27/38; A61L 2/20; B29C 64/106; B29C 64/336; B33Y 70/10

(52) U.S. Cl. CPC B29C 64/255; A61L 27/20; B29C 64/209; A61L 27/3817; A61L 27/3895; B33Y 30/00; A61L 2/206; B29C 64/106; B29C 64/336; B33Y 70/10; A61L 2/208

(57) ABSTRACT

The present solution includes a 3D printer designed to meet clean room requirements. The printer can be configured for 3D bioprinting. The printer can be used to create cellular scaffolds, tissue grafts, and organs. The printer can include a sterile (or clean room-like) environment for the printing of items. The printer can include an enclosure that isolates the manufacturing processes from the external environment and that can be sterilized between printing runs. The solution also includes a printing kit that can be independently sterilized and passed into the system's enclosure.

### CROSS REFERENCE TO RELATED APPLICATIONS

The present application claims priority to and is a National Stage application, filed under 35 U.S.C § 371, of International Application No. PCT/M2018/059476, filed on Nov. 29, 2018, which claims the benefit, under 35 USC § 119(e) of U.S. Provisional Patent Application No. 62/652,757, filed Apr. 4, 2018, and U.S. Provisional Patent Application No. 62/592,202, filed Nov. 29, 2017. Each of the foregoing applications are incorporated herein by reference for all purposes.

### BACKGROUND OF THE DISCLOSURE

Additive manufacturing, or 3D printing, can generally include “printing” an object by successively depositing layers of patterned material atop one another. Additive manufacturing can, in a process termed “bioprinting,” generate biological components or structures that can include cells, proteins, or growth factors that have biological function in the produced construct. The bioprinting process may need to comply with the good manufacturing practices (GMP) guidelines to be applicable to clinical or pharmaceutical use. The printing material can be extruded through the lumen of a printing nozzle. The printing material can be extruded from the printing nozzle under pressure. Extruding the printing material under pressure can cause the formation of aerosol droplets that can contaminate the environment surrounding the printer. The production process consists of certain steps but is not limited to these steps

### SUMMARY OF THE DISCLOSURE

In order to print clinical or pharmaceutical substances, a bioprinting process may need to comply with sterile process requirements and prevent the cross contamination. The present solution describes systems and methods to bioprint cellular constructs or organs in a sterile fashion that substantially prevents cross contamination during the manufacturing process.

During the bioprinting process a number of tests may need to be performed on the cellular construct. Some of the tests that are performed can be destructive and can result in damage to the tested cellular construct or cells therein. The present solution bioprints a plurality of appendices to the cellular construct. The appendices can be printed as removable samples of the cellular construct and can include the same cells and materials as those included within the cellular construct. At predetermined times, the appendices can be removed from the cellular construct and tested. The appendices can serve as a proxy for the cellular construct and provide insight into the health and proliferation of the cells within the cellular construct.

The present solution includes a three-dimensional (3D) printer designed to meet clean room requirements in biologics or pharmaceutical applications. The printer can be configured for 3D bioprinting. The printer can be used to create acellular scaffolds or organ templates, cellular scaffolds, tissue grafts, and multi-cellular organs. For example, the printer can be used to generate cellular tissues such as skin, bone, and cartilage, which can generally be referred to as cellular constructs. These tissue constructs can include biological components such as cells, growth factors, pharmaceuticals, or a combination thereof. The biological components can be mixed, solubilized, or coextruded with synthetic or natural polymers, proteins, or other biocompatible materials. The biological components can be included into the bioprinted materials, often hydrogels, by mixing them before the printing process or alternatively by introducing the biological components after the printing process as coatings or infill. Cells can be embedded in the polymer mix to form biologically functional cellular constructs, tissue grafts, or organs. Cells or other biological components can be introduced to printed scaffold structures or templates by spray coating the object or by infiltration of the biological material into the printed template. The printer can also be used to manufacture custom pharmaceutical tablets and medications.

The produced tissue grafts are produced in sterile environment. Mammalian (e.g., human) cells are isolated from clinical tissue biopsy. The isolated cells can be primary cells, progenitor cells, stem cells or a combination of these. The cells are isolated by mechanically and/or chemically disrupting the extracellular matrix or carrier fluid to release the cells. The collected cells are commonly expanded in monolayer or 3D cultures until a sufficient cell number is obtained. Cells can be expanded multiple weeks depending on the initial isolation cell yield and the application need. Cells can be transfected or gene edited to modify the genome prior printing to obtain desired cell function in the created tissue. These cells can then be mixed with natural or synthetic polymers for a cellular biomaterial mix. Biopolymers such as but not limited to hyaluronan, collagen, gelatin, chondroitin sulfate, alginate, gellan gum or any combination can be used to prepare the polymer mix with the cells. Synthetic polymers such as poly ethylene glycol (PEG), poloxamers, polyoxazolines, polypropylene glycol, poly (L/D)lactide, polyglycolic acid, polymethacrylate polyachrylamide or a combination or a block-copolymers of these. For example, a polymer mix of alginate and gellan gum can be used to mix the cells to for the cellular polymer mix that is suitable for a bioprinting process.

The cellular polymer mix such as alginate and gellan gum with chondrocytes can be loaded into a printing syringe after a mixing process to obtain homogeneous end material. The mixing process can be manual mixing, extrusion with static mixer or an active mixing process which end product is collected to the syringe. Mixing process can be performed inside the syringe or before loading the materials mix into the syringe.

The present solution can also include printing kits. Each printing kit can include printing materials syringe and printer components for a printing run. The printing kit, and the components thereof, can be sterilized and then passed into the printer's enclosure through an airlock. Once in the enclosure, the components can be assembled to form the 3D printer's deposition head and the deposition head can be loaded with the printing materials. Multiple printing syringes and nozzles can be used during the printing process to produce multi-material or multi-cellular constructs. Multiple syringes can be used alternating the extruding syringe within the printing process. The kit can also include a sterile transportation unit into which the completed item is deposited. The transportation unit enables the item to be transferred to an incubator (or other location) while remaining in a sterile environment. The waste from the printing run can be placed back into the kit and removed from the printer, which can be sterilized after the printing run.

The printer can include a sterile (or clean room-like) environment for the printing of items. The printer can include an enclosure that isolates the manufacturing processes from the external environment. The enclosure can be flooded with chemical sterilizers to sterilize the enclosure between printing runs. The enclosure can also prevent the aerosol droplets (generally referred to as particles) from contaminating the external environment. All printer surfaces are compatible with chemical acid and base cleaning cycles and gassing. After printing, the isoprinter can be wiped down with acid and base detergents to remove any possible spills or solid materials preventing the gas to reach all printer surfaces. After the wiping, the gassing utilizing H₂O₂ or similar is performed to sterilize the isoprinter.

The bioprinted construct, composed of liquid, semi-solid or gel-like materials is required to go through a crosslinking process to further stabilize, solidify or reinforce the structure of the created tissue graft. Multiple gelation methods including but not limited to thermal, ionic-, enzymatic, radical or chemical reactions can be used within the isolator space during or after the bioprinting process. For example gellan gum and alginate biopolymer mix can be crosslinked in the presence of mono-, di- or tri-valent cations including but not limited to Mg²⁺, Ca²⁺, Sr²⁺, Ba²⁺, Zn²⁺, Cu²⁺ or Fe³⁺.

According to at least one aspect of the disclosure, an additive manufacturing system can include at least one pass-through chamber coupled with an enclosure. The at least one pass-through chamber can include a first portal to enable passage from an external environment to an interior of the at least one pass-through chamber and a second portal to enable passage from the interior of the at least one pass-through chamber to an interior of the enclosure. The enclosure can include a first port configured to receive a deposition head of a three-dimensional printer. The enclosure can include a first bellow configured to couple with the deposition head and a perimeter of the first port. The system can include a 3D printer.

The system can include a printing kit. The printing kit can include a base plate configured to receive material extruded from the deposition head. The printing kit can include a sleeve that can include a first end configured to couple with the deposition head and a second end configured to couple with the base plate to form a secluded volume within the enclosure.

The printing kit can include a containment bag configured to collect waste from a process of printing a biological scaffold with the 3D printer. The printing kit can include a transport unit configured to enable transportation of the biological scaffold. The printing kit can include syringe including a printing material of the 3D printer. The printing material can include at least one biopolymer and a plurality of cells.

The system can include a second pass-through chamber coupled with the enclosure. The system can include one or more access ports configured to enable a user to manipulate items within the enclosure. The 3D printer can include a plurality of deposition heads. Each of the plurality of deposition heads can be configured to deposit a different printing material.

According to at least one aspect of the disclosure, an additive manufacturing kit can include a base plate configured to receive material extruded from a deposition head of a three-dimensional printer. The kit can include a sleeve that can include a first end configured to couple with the deposition head and a second end configured to couple with the base plate to form a secluded volume. The kit can include a syringe that can include a printing material.

In some implementations, the printing material can include a biopolymer mix with cells. The printing kit can include serializable housing to store the base plate, the sleeve, and the syringe.

According to at least one aspect of the disclosure, a method can include isolating chondrocytes from a biopsy. The method can include generating a biopolymer printing material that can include at least one polymer and the chondrocytes. The method can include forming a cellular construct from the biopolymer printing material with an additive manufacturing system. The system can include at least one pass-through chamber coupled with an enclosure. The at least one pass-through chamber can include a first portal to enable passage from an external environment to an interior of the at least one pass-through chamber and a second portal to enable passage from the interior of the at least one pass-through chamber to an interior of the enclosure. The enclosure can include a first port configured to receive a deposition head of a three-dimensional printer. The enclosure can include a first bellow configured to couple with the deposition head and a perimeter of the first port.

The biopolymer printing material can include at least one of a gelling polysaccharide or sodium alginate. The method can include forming at least one appendix on the cellular construct from the biopolymer printing material. The method can include excising one of the at least one appendices of the cellular construct for testing. The method can include cross linking the cellular construct with a calcium chloride solution.

The biopolymer printing material can include at least one of differentiated progenitor cells or differentiated stem cells harvested from the biopsy. The method can include culturing the chondrocytes from the biopsy until the chondrocytes reach a predetermined cell count. The method can include sterilizing the enclosure.

### BRIEF DESCRIPTION OF THE DRAWINGS

The accompanying drawings are not intended to be drawn to scale. Like reference numbers and designations in the various drawings indicate like elements. For purposes of clarity, not every component may be labeled in every drawing. In the drawings:

FIGS. 1A and 1B illustrate different views of an example isolation printer that can be used to manufacture cellular constructs.

FIG. 2 illustrates a schematic of an example kit for use with the isolation printer illustrated in FIGS. 1A and 1B.

FIG. 3 illustrates a block diagram of an example method to bioprint a cartilage organ using the system illustrated in FIGS. 1A and 1B.

FIG. 4 illustrates a block diagram of an example method for additive manufacturing using the isolation printer illustrated in FIGS. 1A and 1B.

FIG. 5 illustrates an example cellular construct with removable appendices manufactured with the example isolation printer illustrated in FIGS. 1A and 1B.

### DETAILED DESCRIPTION

The various concepts introduced above and discussed in greater detail below may be implemented in any of numerous ways, as the described concepts are not limited to any particular manner of implementation. Examples of specific implementations and applications are provided primarily for illustrative purposes.

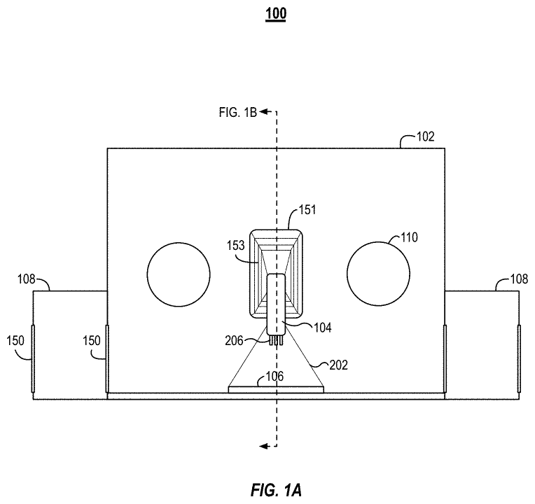

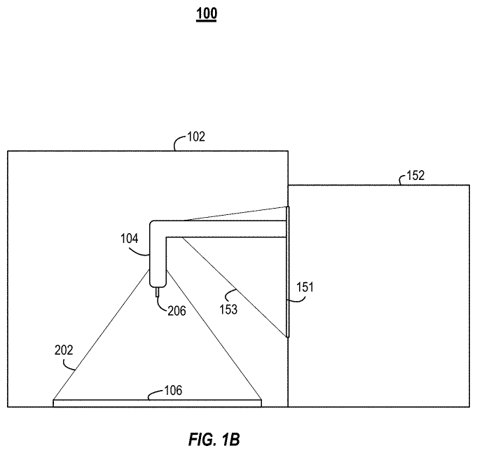

FIGS. 1A and 1B illustrate an example isolation printer 100, which can also be referred to as an isoPrinter 100. The isoPrinter 100 can be an additive manufacturing system. FIG. 1A illustrates a front view of the isoPrinter 100. FIG. 1B illustrates a cross-sectional side view of the isoPrinter 100.

The isoPrinter 100 includes an enclosure 102 that houses a deposition head 104 of a 3D printer 152. The deposition head 104 can also be referred to as a printing head. In some implementations, the isoPrinter 100 can include a plurality of deposition heads 104. A printing nozzle 206 can be coupled with each of the deposition heads 104. The different deposition heads 104 can be used to print multi-material or multi-cellular tissue grafts. For example, the isoPrinter 100 can print an internal support structure from a first material and a cellular construct around the internal support structure in a second material.

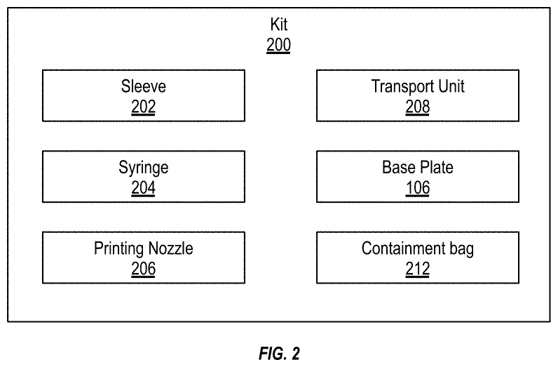

The isoPrinter 100 can include a base plate 106 onto which the deposition head 104 can deposit printing material. The enclosure 102 can include a plurality of airlocks 108 (or pass-through chambers 108). In some implementations, one airlock 108 can be used to pass materials and the kit described in relation to FIG. 2 into the enclosure 102 and a second airlock 108 can be used to remove materials and the kit from the enclosure 102. Each of the airlocks 108 can include one or more portals 150. A first portal 150 can enable passage from the exterior of the system 100 to the interior of the airlock 108. The airlock 108 can be coupled with the enclosure 102. A second portal 150 can enable passage between the interior of the airlock 108 and the interior of the enclosure 102. For example, the below described kit can be passed into the airlock 108 via the first portal 150 and then passed into the interior of the enclosure 102 via the second portal 150. The enclosure 102 can also include access ports 110 that enable a user to manipulate materials and items within the enclosure 102.

The enclosure 102 can isolate the manufacturing process performed by the deposition head 104 from the external environment. By isolating the manufacturing process, the isoPrinter 100 can be used in cleanroom environments. For example, the isoPrinter 100 can be used in class B-C clean rooms. The walls of the enclosure 102 can include acrylic glass or other transparent materials that can be sterilized, disinfected, and/or sanitized. In some implementations, a substantial portion of each of the walls can be transparent. In other implementations, the walls of the enclosure 102 can be constructed from metal and the walls can include transparent viewing ports.

The enclosure 102 can be sealed to prevent airflow between the interior and exterior of the enclosure 102. In some implementations, the isoPrinter 100 can include a pump to positively or negatively pressurize the interior of the enclosure 102. In some implementations, the environment inside the enclosure 102 can be controlled for desired temperature, atmospheric gas and humidity.

The interior of the enclosure 102 can be sterilized. The enclosure 102 can include inlet and outlet ports to enable the introduction of sterilizers by a sterilization unit. For example, after use, the interior of the enclosure 102 can be flooded with H₂O₂ gas to sterilize the enclosure 102.

The enclosure 102 can include a port for the deposition head 104. For example, the controller and other components of the 3D printer can be positioned outside the enclosure 102. The 3D printer's deposition head 104 can pass into the enclosure 102 through the port. The port can include a rubber bellow that couples to the deposition head 104 and forms a seal between the deposition head 104 and the perimeter of the port. The bellow can enable the deposition head 104 to freely move in an x, y, and z directions within the enclosure 102.

In some implementations, the deposition head 104 can include positioning and feedback sensors. The sensors can be configured to determine the location of the deposition head 104 (and the printing nozzle) within the enclosure 102 and relative to the base plate 106 and the material already printed on the base plate 106. The sensors can include piezoelectric sensors and laser-based distance sensors.

The enclosure 102 can also include airlocks 108. The airlocks 108 can enable a user to pass materials and equipment into and out of the enclosure 102 without cross contaminating the isoPrinter 100 and the environment by substantially preventing contaminants or undesirable particles from passing between the interior and exterior of the enclosure 102. For example, an airlock 108 can include an interior and an exterior door (e.g., portals 150). The interior door can face the interior of the enclosure 102 and the exterior door can face the external environment. A user can first open the exterior door, with the interior door closed, and place the materials within the interior of the airlock 108. After shutting the exterior door, the user can open the interior door (via the gloves of the access port 110). The airlocks 108 can include air showers to flow air over the items within the airlocks 108 to remove particles and contaminants from the items. In some implementations, the airlocks 108 can flow a gas sterilizer into the airlock 108 to sanitize or sterilize the item within the airlock 108.

The access ports 110 can include openings in the wall of the enclosure 102. Gloves can be sealed to the access ports 110 to enable a user to manipulate items within the enclosure 102 while still providing a barrier between the internal and external environment of the enclosure 102. In some implementations, the access port 110 can include bellows that enable tools or robotic arms to be used within the enclosure 102.

The deposition head 104 can include the of extruder of the 3D printer. The deposition head 104 can include a printing nozzle through which the printing material is extruded. The printing material can include plastics, metals, synthetic polymers, or other biocompatible materials. In some implementations, printing nozzle can be removable. The printing nozzle can include brass, stainless steel, hardened steel, or plastic. The printing material can be passed through the deposition head 104 under pressure and at a controlled temperature to the printing nozzle. The printing material can be extruded from the deposition head 104 through a lumen in the printing nozzle. In some implementations, the printing nozzle can be the needle of a syringe. In these implementations, a filled syringe can be inserted into the deposition head 104. The deposition head 104 can include an actuator that presses against a plunger of the syringe and causes printing material to be extruded or a screw-based system to move material in the threads from the syringe's needle.

The isoPrinter 100 can include a port 151. The port 151 can be opening in the enclosure 102 in the wall through which the deposition head 104 extends from the 3D printer 152. The isoPrinter 100 can include a bellow 153 that can couple with a perimeter of the port 151 on a first end of the bellow 153 and the deposition head 104 on the second end of the bellow 153. The bellow 153 can enable the deposition head 104 to move freely within the port 151. The bellow 153 can form a seal to prevent contaminants from passing through the port 151 and into the interior of the enclosure 102.

The isoPrinter 100 can include a base plate 106. The deposition head 104 can deposit material onto the base plate 106. The base plate 106 can be coupled to one or more actuators to enable the base plate 106 to move in the x, y, and z directions. The base plate 106 can be a component of the kit described in relation to FIG. 2. For example, prior to each build, a base plate 106 can be passed into the enclosure 102 and secured to the actuators. In some implementations, the base plate 106 is static and only the deposition head 104 moves.

FIG. 2 illustrates a schematic of an example kit 200. The kit 200 can include the materials, tools, and other items that are used for a specific manufacturing run. In some implementations, the kit 200 can include any combination of a sleeve 202, a syringe 204, a printing nozzle 206, a transport unit 208, a base plate 106, or a containment bag 212. The kit 200 includes a housing to store the components of the kit. The kit housing and the components of the kit 200 can be sterilized.

The sleeve 202 can be a flexible bellow, tube, or skirt. A first end of the sleeve 202 can couple with the deposition head 104 and a second end of the sleeve 202 can couple with the perimeter of the base plate 106. When sealed between the deposition head 104 and the base plate 106, the sleeve 202 can form a secluded volume in which the printing deposition takes place. Use of the sleeve 202 can confine contaminants and particles from dispersing from the deposition head 104 and throughout the enclosure 102. Containment of the particles can make the sterilization and cleaning of the enclosure 102 easier, quicker, and more cost effective. The sleeve 202 can be configured to enable full freedom of movement of the deposition head 104 during the manufacturing process. The sleeve 202 can be plastic-, rubber-, or silicon-based. The sleeve 202 can include a seal, such as a gasket or O-ring, at each of its ends to form a hermetical seal between the deposition head 104 and the base plate 106. In some implementations, the kit 200 can include clips, lugs, or locks that can be used to secure the sleeve 202 to the deposition head 104 and/or the base plate 106.

The kit 200 can include one or more printing nozzles 206. Each of the different printing nozzles 206 can include different lumen diameters for the extrusion of the printing material. The smaller diameter lumens can enable the 3D printer to print with a relatively higher resolution when compared to larger diameter lumens. For each run, the printing nozzle 206 can be replaced.

The kit 200 can also include a syringe 204. The syringe 204 can be prefilled with a biopolymer mix with or without cells or other printing material. A user can use the syringe 204 to fill the deposition head 104 with the printing material. In some implementations, the syringe 204 can be placed directly into the deposition head 104 and the syringe's needle can be used as the printing nozzle. The kit 200 can include a plurality of different syringes 204. Each of the different syringes 204 can be filled with a different (or additional) printing material.

The kit 200 can include a transport unit 208. The transport unit 208 can be sterile container into which the printed item is directly created or placed once printed. The printed item can be placed in the transport unit 208, passed to the exterior of the enclosure 102, via an airlock 108, and then transported to another location where further processing can be performed on the printed item. In some implementations, when a biological scaffold, such as for an ear or other cellular construct, is printed, the printed item can be taken from the isoPrinter 100, via the transport unit 208, to an incubator where the cells in the biological scaffold can be incubated. In some implementations, the cells can be incorporated into the biopolymer mix and printed on to a biological scaffold. In other implementations, the cells can be seeded onto the biological scaffold after the scaffold is printed. In some implementations, the transport unit 208, with the printed item, can be sterilized before implantation into the patient.

The kit 200 can also include a containment bag 212. Once the printing run is completed, the waste and disposable items of the kit 200 can be placed in the containment bag 212 and the containment bag 212 can be sealed. For example, the sleeve 202, syringe 204, and printing nozzle 206 can be disposed after each run. In some implementations, the waste material can be directly placed into the housing of the kit 200 and not into a containment bag 212.

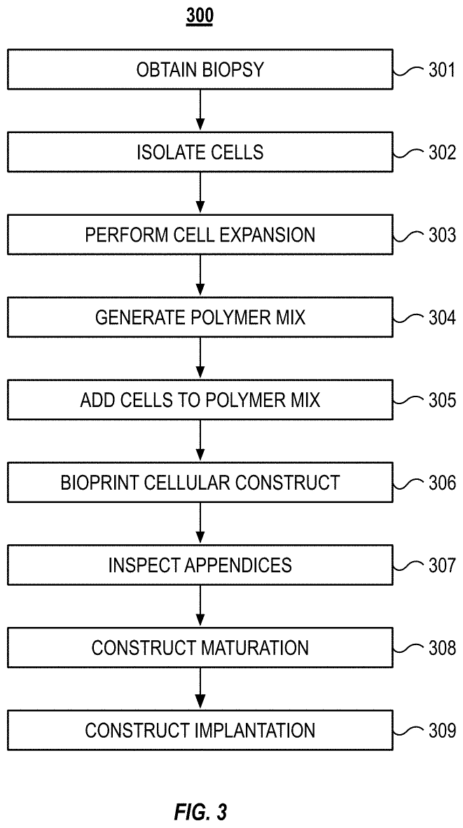

FIG. 3 illustrates a block diagram of an example method 300 to bioprint a cartilage organ. For example, the method 300 can be used to manufacture an ear or a nose. The method 300 can include obtaining a biopsy (step 301). The method 300 can include isolating cells from the biopsy (step 302). The method 300 can include expanding the cells by supporting multiple cell doublings until sufficient number of cells is obtained (step 303). The method 300 can include generating a polymer mix (step 304) and adding the cells to the polymer mix (step 305). The method 300 can include bioprinting the cellular construct (step 306) and inspecting the construct's appendices (step 307). The method 300 can include further culturing of the construct to form matured tissue (step 308). The method 300 can include the implantation of the construct (step 309).

As set forth above, the method 300 can include obtaining a biopsy (step 301). The biopsy can be obtained from the patient into which the organ will eventually be implanted. In some implementations, the biopsy can be obtained from a donor. The biopsy can include auricular cartilage, nasal cartilage, nucleus pulposus, meniscus, trachea, nasal cartilage, rib cartilage, articular cartilage, synovial fluid, vitreous humor, brain, spinal cord, muscle, connective tissues, small intestinal submucosa, or liver tissue. For manufacturing an ear, the biopsy can be a 4-8 mm diameter, full thickness circular cartilage biopsy sample that is obtained from the ear contralateral to the microtia ear. Once resected, the biopsy can be stored in phosphate-buffered saline (PBS) with gentamicin (50 g/mL). This biopsy is not a critical size defect and will heal over time. In some instances, the tissue biopsy can be transported in 2-8° C. to enhance the cells ability to survive before cells isolation.

The method 300 can include isolating cells from the biopsy (step 302). The cells can be chondrocytes. The connective tissue or other unwanted tissue can be removed from the biopsy tissue and the sample tissue (e.g., cartilage) can be minced. The tissue can be minced to a size of between about 5 μm and about 50 μm, between about 50 μm and about 200 μm, or between about 200 μm and about 100 μm. Sterile filtered digestion medium including DMEM and Ham's F12, 10% fetal bovine serum (FBS), and collagenase 0.66 units/mL enzyme can be combined with the minced cartilage and allowed to incubate for 16-18 hours at 37° C. under static conditions. This can create a suspension of released chondrocytes. The suspension of released chondrocytes can be passed through a 100 μm cell strainer and centrifuged. The pelleted chondrocytes can be resuspended in fresh sterile DMEM+Ham's F12 supplemented with 10% FBS and 25 μg/mL ascorbic acid. In some implementations, the total number of cells are counted, and cell viability is determined via Trypan blue staining.

The method 300 can include expanding the cells (step 303). The cells (e.g., the chondrocytes) can be seeded into culture flasks at a concentration of about 3000 cells/cm′. The cells can be seeded in densities between about 100 and about 1000 cells/cm′ or between about 1000 and about 10000 cells/cm′ can be used according to a specific cell requirements. In some implementations, the cells can be cryopreserved for ease of patient scheduling or transportation. If cryopreserved, the cells can be suspended in a medium that can include DMEM and Ham's F12, 10% FBS, 10% DMSO. The cells can be cooled at a controlled rate of 2° C. per minute until −80° C. prior to storage in liquid or vaporized liquid nitrogen. Once removed from cryopreservation (e.g., once the patient is scheduled for the implantation procedure), the cells are thawed, and cell expansion is continued until approximately 70-100×10⁶ cells are present. The cells can be harvested and washed with FBS-free medium. In some implementations, a portion of the cells are harvested, and the cell viability and gene expression are measured. Progenitor and stem cells can be differentiated at this stage prior mixing with biopolymers or used otherwise to guarantee target tissue formation. Differentiated, unipotent, cells can be further mixed into the biopolymers or used in the scaffold coatings.

The method 300 can include generating a polymer mix (step 304). The polymer mix can be prepared by mechanically mixing gellan gum (35 mg/mL) and sodium alginate (25 mg/mL) with dextran solution (osmolarity 300 mOsmol) at 90° C. The polymer mixture can be aseptically stored at room temperature in syringes for later use with cells. In some implementations, other gelling polysaccharides can be used for the gellan gum or alginate. For example, guar gum, cassia gum, konjac gum, Arabic gum, ghatti gum, locust bean gum, xanthan gum, xanthan gum sulfate, carrageen, carrageen sulfate, or any mixture thereof can be used. In some implementations, the polymer mix can include a bioresorbable polymer, such as PLA (polylactic acid or polylactide), DL-PLA (poly(DL-lactide)), L-PLA (poly(L-lactide)), polyethylene glycol (PEG), PGA (polyglycolide), PCL (poly-ecaprolactone), PLCL (Polylactide-co-e-caprolactone), dihydrolipoic acid (DHLA), chitosan. In some implementations, the polymer can include a synthetic polymer, such as, polyethylene glycol, polypropylene glycol, polaxomers, polyoxazolines, polyethylenimine, polyvinyl alcohol, polyvinyl acetate, polymethylvinylether-co-maleic anhydride, polylactide, poly-N-isopropylacrylamide, polyglycolic acid, polymethylmethacrylate, polyacrylamide, polyacrylic acid, and polyallylamine. In some implementations, the polymer can include any combination of the above.

The method 300 can include adding the cells to the polymer mix (step 305). A suspension including the cells is added to the polymer mixture generated at step 205 at a 1:10 ratio (cell medium:polymers). The cells can be added via static mixing that is connected directly to a printing syringe to obtain a cell concentration of 6-9×10⁶ cells/ml in the biopolymer mix.

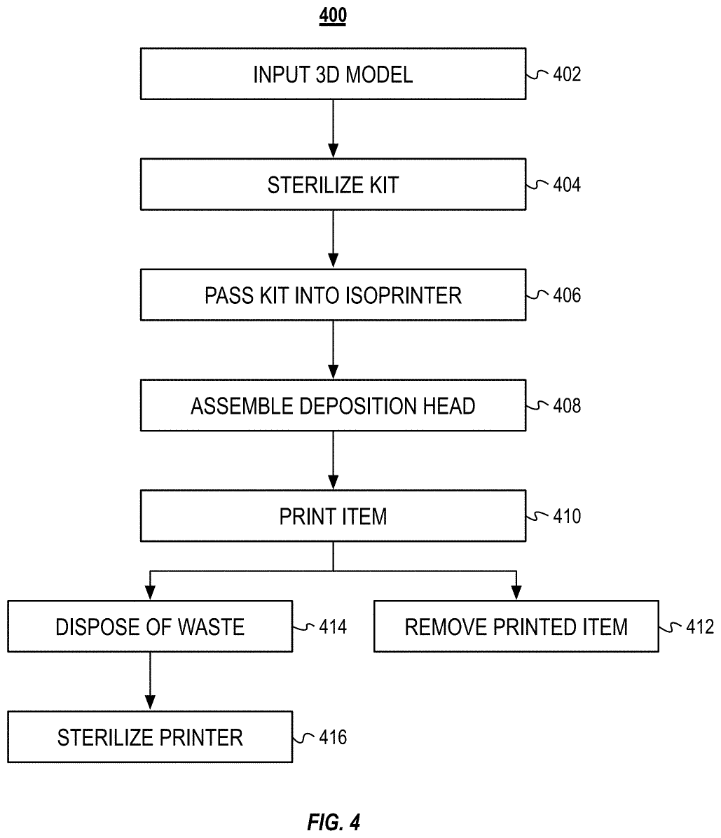

The method 300 can include bioprinting the cellular construct (step 306). The method to bioprint the cellular construct is discussed further in relation to FIG. 4. As an overview, a printing syringe filled with the biopolymer mix formed in step 305 can be brought to the printer via pass box as part of the prepared printing kit. The printing syringe can be attached to or inserted in the deposition head or syringe holder of the printer. In some implementations, the printing syringe can be used to fill a reservoir in the deposition head with the biopolymer mix. In an additive fashion, the biopolymer can be extruded form the printing syringe for form the cellular construct. Secondary polymer mix can be extruded from a parallel syringe for three-dimensional support structures. The transient support structures can be removed after the construct finish by physical or chemical process such as but not limited to hydrolytic dissolving, pH change or temperature shift.

The method 300 can include inspecting the cellular construct's appendices (step 307). The inspection of the appendices can include removing one or more of the appendices at predetermined intervals. Inspecting the appendices can include performing tests on the excised appendix. The testing can be destructive or non-destructive. The tests can be tests of mechanical and/or biological properties such as, but not limited to, cell viability, gene expression and cell distribution in the cellular construct. The removal and testing of the appendices can occur during a distinct phase of the method 300 or can also occur during the maturation of the construct (step 308, below). For example, the appendices can be periodically removed and tested during the maturation of the construct to determine when the construct is ready for release to the patient.

The method 300 can include construct maturation (step 308). For example, after the cellular construct is manufactured, the cellular construct can be removed from the printer to avoid any possible cross contamination issues. The cellular construct can be cross-linked by applying a calcium chloride solution to the cellular construct. The calcium chloride solution can be applied for 240 minutes while cellular construct is positioned on an agitator plate to prevent local concentration gradient formation in the cross-linking. In some implementations, the cross-linking agent can be a monovalent, divalent and trivalent cation, enzyme, hydrogen peroxide, horseradish peroxidase, radiation polymerizable monomers such as lithium phenyl-2,4,6-trimethylbenzoylphosphinate. Polymers crosslinking via photoinitiators can be crosslinked layer-by-layer during the printing process to allow more homogeneous layer exposure. After crosslinking, despite the technique, the cellular construct can be cultured for about 2-5 weeks in culture medium containing 10 ng/mL of recombinant human transforming growth factor beta three (rhTGF-β3) or other mitogenic growth factor known to affect positively to the used cells. Maturation culture can be done in an incubator or a specific bioreaction can be used to stimulate the maturation via mechanical, chemical or biological stimulation. After the tissue maturation the cellular construct can be washed three times with rhTGF-β3-free medium.

In some implementations, once the cellular construct is cross-linked, the cellular construct can be further measured with reference to the 3D model of the expected cellular construct shape and size. The above procedure can also be performed on the remaining test samples. The test samples can then be tested for cell viability, PCR gene expression, and mechanical properties. Sterility, endotoxin, and Mycoplasma assays are performed on the test samples. The cellular construct can be packed in a sterile container with nutrient medium (e.g., DMEM and Ham's F12 alone), and then shipped to the clinic in a specially designed container that guarantees sterility and nutrition supply for the shipped living construct.

The method 300 can include construct implantation (step 309). The method 300 can include shipment of the construct to clinical site before implantation. The construct can be shipped to the clinical site in a transport unit. The transport unit can maintain a sterile environment until the construct is removed for implantation into a patient.

FIG. 4 illustrates a block diagram of an example method 400 for additive manufacturing that can be used in the above-described method 300. The method 400 can include inputting a 3D model into the isoPrinter (step 402). The method 400 can include sterilizing the kit (step 404). The method 400 can include passing the kit into the isoPrinter (step 406). The method 400 can include assembling the deposition head (step 408). The method 400 can include printing the item (step 410). The method 400 can include removing the printed item from the isoPrinter (step 412). The method 400 can include disposing of the waste (step 414) and sterilizing the isoPrinter (step 416).

Also referring to FIGS. 1 and 2, and as set forth above, the method 400 can include inputting a 3D computer model into the isoPrinter 100 (step 402). The computer model can be generated via a computer aided design (CAD) program. In some implementations, the computer model can be generated by optically scanning a physical model to generate a digital model. The file, including the 3D geometry of the item to be printed item, can be loaded into the isoPrinter 100 via a direct connection (e.g., with a flash drive) or over a network.

The method 400 can include sterilizing the kit 200 (step 404). As discussed above, the kit 200 can include printing materials and disposable or reusable components of the deposition head 104. For example, the kit 200 can include a sleeve 202, one or more syringes 204 and printing nozzles 206, a transport unit 208, a base plate 106, and a containment bag 212. The kit 200 can also include printing filament and/or the syringe 204 can be loaded with printing material, such as a bioink or biopolymer mix. Once the kit 200 is assembled for the printing run, the kit 200 can be sterilized. The kit 200, and the components therein, can be sterilized with heat sterilization, chemical sterilization, radiation sterilization, or any combination thereof.

The method 400 can include passing the kit 200 into the isoPrinter 100 (step 406). The kit 200 can be passed into the enclosure 102 via an airlock 108. Once in the enclosure 102, the components of the kit 200 can be used to assemble the deposition head 104 (step 408). Assembling the deposition head 104 can include loading the syringe 204 or printing material into the deposition head 104. The printing nozzle 206 can also be applied to the deposition head 104. The base plate 106 can be secured to the floor (or actuators in the floor) of the enclosure 102. The sleeve 202 can be coupled with the deposition head 104 and the base plate 106 to form a secluded volume where the additive manufacturing process is performed. In some implementations, the method 400 does not include the use of the sleeve 202. The 3D printer can then print the item (step 410).

Once the print run is complete, the printed item can be removed from the isoPrinter 100 (step 412). In some implementations, before removing the printed item from the isoPrinter 100, the printed item can be placed in a transport unit 208. The transport unit 208 can maintain the printed item in a sterile environment until the printed item is further processed (e.g., placed in an incubator or crosslinked) or implanted into a patient. In some implementations, the transport unit 208 and printed item can be re-sterilized before implantation or further processing. The printed item can be removed from the enclosure 102, in the transport unit 208, via one of the enclosures airlocks 108.

Once the run is complete, the user can dispose of the waste (step 414). The waste can include the used sleeve 202, syringe 204, base plate 106, and printing nozzle 206. The user can place the used items into the containment bag 212. The user can pass the containment bag 212 out of the isoPrinter 100 via an airlock 108. The used components can then be discarded or cleaned, sterilize, and reused. For example, sleeve 202 can be steam cleaned and reused in a subsequent printing run. The interior of the enclosure 102 can then be sterilized (step 416). The interior of the enclosure 102 can be chemically sterilized or heat sterilized with, for example, steam. In an example of chemical sterilization, the enclosure 102 can be flooded with ethylene oxide or hydrogen peroxide gas. In some implementations, the isoPrinter 100 can be re-calibrated for the next print run.

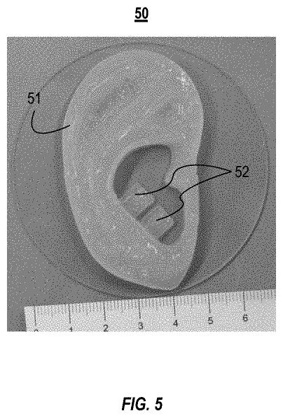

FIG. 5 illustrates an example construct 50 manufactured with removable appendices 52. The construct 50 can include a body 51 and one or more appendices 52. The body 51 can include the part of the construct 50 that forms the final part that is implanted into the patient. For example, the body 51 can include the ear, nose, or other part that is delivered to the patient. The appendices 52 can be coupled with or can extend from the body 51.

The construct 50 can be manufactured with one or more appendices 52. The appendices 52 can be distributed across one or more edges of the construct 50. For example, the appendices 52 can be placed at locations around the edge of the body 51 where removal of the appendices 52 will do minimal or no physical or cosmetic damage to the body 51 when the appendices 52 are removed. In some implementations, the appendices 52 can be printed onto the print platform (on which the body 51 is printed) and are not coupled to the body 51. The appendices 52 can be manufactured from the same material as the body 51. The appendices 52 can include internal support structures or other components that are incorporated into body 51. The appendices 52 can be configured to match the mechanical and biological properties of the body 51. Being manufactured from the same material as the body 51 (and having the same properties as the body 51), the appendices 52 can act as proxies for the body 51 during pre-release testing.

For example, the construct 50 can be printed from a polymer matrix that includes a mixture of cells. Once the construct 50 is 3D printed, the construct 50 can be incubated to enable the cells to mature and multiply. The appendices 52 can be sequentially removed from the body 51 at different time points along the maturation process. At each of the different time points, the removed appendix 52 can be tested to determine, for example, cell differentiation, cellular density, mechanical properties of the cells, cell viability, gene expression, cell distribution in the cellular construct 50, drug interaction testing, or any combination thereof.

The appendices 52 can have a surface area between about 10 mm² and about 500 mm², between about 50 mm² and about 1000 mm², between about 100 mm² and about 750 mm², between about 200 mm² and about 500 mm², or between about 300 mm² and about 500 mm².

While operations are depicted in the drawings in a particular order, such operations are not required to be performed in the particular order shown or in sequential order, and all illustrated operations are not required to be performed. Actions described herein can be performed in a different order.

The separation of various system components does not require separation in all implementations, and the described program components can be included in a single hardware or software product.

Having now described some illustrative implementations, it is apparent that the foregoing is illustrative and not limiting, having been presented by way of example. In particular, although many of the examples presented herein involve specific combinations of method acts or system elements, those acts and those elements may be combined in other ways to accomplish the same objectives. Acts, elements and features discussed in connection with one implementation are not intended to be excluded from a similar role in other implementations or implementations.

The phraseology and terminology used herein is for the purpose of description and should not be regarded as limiting. The use of “including” “comprising” “having” “containing” “involving” “characterized by” “characterized in that” and variations thereof herein, is meant to encompass the items listed thereafter, equivalents thereof, and additional items, as well as alternate implementations consisting of the items listed thereafter exclusively. In one implementation, the systems and methods described herein consist of one, each combination of more than one, or all of the described elements, acts, or components.

As used herein, the term “about” and “substantially” will be understood by persons of ordinary skill in the art and will vary to some extent depending upon the context in which it is used. If there are uses of the term which are not clear to persons of ordinary skill in the art given the context in which it is used, “about” will mean up to plus or minus 10% of the particular term.

Any references to implementations or elements or acts of the systems and methods herein referred to in the singular may also embrace implementations including a plurality of these elements, and any references in plural to any implementation or element or act herein may also embrace implementations including only a single element. References in the singular or plural form are not intended to limit the presently disclosed systems or methods, their components, acts, or elements to single or plural configurations. References to any act or element being based on any information, act or element may include implementations where the act or element is based at least in part on any information, act, or element.

Any implementation disclosed herein may be combined with any other implementation or embodiment, and references to “an implementation,” “some implementations,” “one implementation” or the like are not necessarily mutually exclusive and are intended to indicate that a particular feature, structure, or characteristic described in connection with the implementation may be included in at least one implementation or embodiment. Such terms as used herein are not necessarily all referring to the same implementation. Any implementation may be combined with any other implementation, inclusively or exclusively, in any manner consistent with the aspects and implementations disclosed herein.

The indefinite articles “a” and “an,” as used herein in the specification and in the claims, unless clearly indicated to the contrary, should be understood to mean “at least one.”

References to “or” may be construed as inclusive so that any terms described using “or” may indicate any of a single, more than one, and all of the described terms. For example, a reference to “at least one of ‘A’ and ‘B’” can include only ‘A’, only ‘B’, as well as both ‘A’ and ‘B’. Such references used in conjunction with “comprising” or other open terminology can include additional items.

Where technical features in the drawings, detailed description or any claim are followed by reference signs, the reference signs have been included to increase the intelligibility of the drawings, detailed description, and claims. Accordingly, neither the reference signs nor their absence have any limiting effect on the scope of any claim elements.

The systems and methods described herein may be embodied in other specific forms without departing from the characteristics thereof. The foregoing implementations are illustrative rather than limiting of the described systems and methods. Scope of the systems and methods described herein is thus indicated by the appended claims, rather than the foregoing description, and changes that come within the meaning and range of equivalency of the claims are embraced therein.

What is claimed:

1. An additive manufacturing system comprising:
a three-dimensional (3D) printer comprising a deposition head configured to extrude a biopolymer printing material;
an enclosure configured to maintain a sterile environment, the enclosure comprising:
a first port configured to receive the deposition head of the 3D printer;
a first bellow configured to couple with the deposition head and a perimeter of the first port; and
at least one pass-through chamber coupled with the enclosure, the at least one pass-through chamber comprising a first portal to enable passage from an external environment to an interior of the at least one pass-through chamber and a second portal to enable passage from the interior of the at least one pass-through chamber to an interior of the enclosure.

2. The system of claim 1, further comprising a printing kit comprising:
a base plate configured to receive material extruded from the deposition head; and
a sleeve comprising a first end configured to couple with the deposition head and a second end configured to couple with the base plate to form a secluded volume within the enclosure.

3. The system of claim 2, wherein the printing kit further comprises:
a containment bag configured to collect waste from a process of printing a biological scaffold with the 3D printer; and
a transport unit configured to enable transportation of the biological scaffold.

4. The system of claim 1, further comprising a printing kit comprising a syringe including a printing material of the 3D printer.

5. The system of claim 4, wherein the printing material comprises at least one biopolymer and a plurality of cells.

6. The system of claim 1, further comprising a second pass-through chamber coupled with the enclosure.

7. The system of claim 1, one or more access ports configured to enable a user to manipulate items within the enclosure.

8. The system of claim 1, wherein the 3D printer comprises a plurality of deposition heads.

9. The system of claim 8, wherein each of the plurality of deposition heads is configured to deposit a different printing material.

10. An additive manufacturing kit comprising:
a base plate configured to receive material extruded from a deposition head of a three-dimensional (3D) printer;
a sleeve comprising a first end configured to couple with the deposition head and a second end configured to couple with the base plate to form a secluded volume; and
a syringe comprising a printing material.

11. The kit of claim 10, wherein the printing material is a biopolymer mix with cells.

12. The kit of claim 10, further comprising a sterilisable housing to store the base plate, the sleeve, and the syringe.

13. A method comprising:
isolating chondrocytes from a biopsy;
generating a biopolymer printing material comprising at least one polymer and the chondrocytes;
forming a cellular construct from the biopolymer printing material with an additive manufacturing system comprising:
at least one pass-through chamber coupled with an enclosure, the at least one pass-through chamber comprising a first portal to enable passage from an external environment to an interior of the at least one pass-through chamber and a second portal to enable passage from the interior of the at least one pass-through chamber to an interior of the enclosure;
the enclosure comprising:
a first port configured to receive a deposition head of additive manufacturing system; and
a first bellow configured to couple with the deposition head and a perimeter of the first port.

14. The method of claim 13, wherein the biopolymer printing material comprises at least one of a gelling polysaccharide or sodium alginate.

15. The method of claim 13, further comprising forming at least one appendix on the cellular construct from the biopolymer printing material.

16. The method of claim 15, further comprising excising one of the at least one appendices of the cellular construct for testing.

17. The method of claim 13, further comprising cross linking the cellular construct with a calcium chloride solution.

18. The method of claim 13, wherein the biopolymer printing material comprises at least one of differentiated progenitor cells or differentiated stem cells harvested from the biopsy.

19. The method of claim 13, further comprising culturing the chondrocytes from the biopsy until the chondrocytes reach a predetermined cell count.

20. The method of claim 13, further comprising sterilizing the enclosure.
